(19) United States
(12) Patent Application Publication
(10) Pub. No.: US 2025/0135068 A1
(43) Pub. Date: May 1, 2025

(54) REGENERATIVE TISSUE REPAIR PRODUCT DERIVED FROM DECELLULARIZED ANIMAL LIVER AND METHOD OF MAKING

(71) Applicant: RTT MEDICAL, INC., Orlando, FL (US)

(72) Inventors: Rodney W. Bosley, JR., Orlando, FL (US); Nico Raices, Saint Cloud, FL (US); Michelle D. Kofron, Palm Beach Gardens, FL (US); Josh S. Copus, Winston-Salem, NC (US)

(21) Appl. No.: 18/907,639

(22) Filed: Oct. 7, 2024

Related U.S. Application Data
(60) Provisional application No. 63/592,936, filed on Oct. 25, 2023.

(51) Int. Cl. A61L 27/36; A61L 27/24; A61L 27/50

(52) U.S. Cl. CPC A61L 27/3691; A61L 27/24; A61L 27/3633; A61L 27/3687; A61L 27/50; A61L 2430/34; A61L 2430/40

(57) ABSTRACT

A regenerative tissue repair product and method of making includes a decellularized extracellular matrix derived from animal liver. The decellularized extracellular matrix retains the collagen morphology of the animal liver prior to decellularization and having less than 500 ng DNA/mg dry extracellular matrix, greater than 760 microgram total collagen per milligram, the collagen comprising soluble and insoluble collagen, and greater than 1.6 μg glycosaminoglycans per milligram of dry extracellular matrix.

### PRIORITY APPLICATION(S)

This application is based upon U.S. provisional patent Application No. 63/592, 936 filed Oct. 25, 2023, the disclosure which is hereby incorporated by reference in its entirety.

### FIELD OF THE INVENTION

The present invention relates to the field of tissue repair and wound care, and more particularly, this invention relates to regenerative tissue material derived from the extracellular matrix of decellularized animal tissue used for tissue repair and wound care and associated methods of making.

### BACKGROUND OF THE INVENTION

Tissue compositions used for soft tissue repair, wound care and similar applications are usually made from the extracellular matrix of decellularized animal organs and tissues and used to restore damaged or missing tissues in patients where the wounds or injuries do not heal adequately. Example wounds include partial or full-thickness wounds, trauma abrasions, lacerations, surgical wounds, burns, and vascular ulcers. These wounds can be problematic and require tissue grafts to help restore the damaged or missing tissues in patients.

These products may be made from the extracellular matrix (ECM) derived from decellularized animal tissues. The ECM is the scaffolding that usually occupies the space between cells and is part of every organ in a species. ECM is xenogenically transferable from one species to another, and thus, universal among species. The ECM is usually an interstitial matrix and a complex infrastructure. The ECM, depending on where it is derived, includes collagen, proteoglycans, glycoproteins, laminin, fibronectin, elastin, and other growth factors. The ECM mediates cell-to-cell interactions and proliferates and differentiates similar cells and may modulate biological activities of growth factors.

A regenerative tissue material or other tissue graft product derived from decellularized ECM may be used to repair the tissues, organs and anatomic sites in the body that are different from the tissue or organs from which the ECM derived. However, not all ECM materials provide sufficient structural integrity and bioactivity to aid in healing a wound. Animal liver has been found to be a beneficial organ from which the extracellular matrix may be derived since the liver has a natural honeycomb collagen structure that includes hexagonal cords of hepatocytes that are the principal parenchymal cells. The ECM derived from decellularized liver is an excellent structure for regenerative tissue, such as in a sheet form for wound care. It is difficult, however, to remove the cells from the ECM using typical chemicals in a decellularization process. The honeycomb collagen structure in the liver maintains hold of the cells during the decellularization process.

During decellularization, it is possible to increase the amount of chemicals and application time of the chemicals, for example, but those harsh chemical processes will remove most of the proteins and reduce the effectiveness of the ECM for surgical repair and reconstruction of different tissues. Other techniques for producing a regenerative tissue material derived from the ECM of decellularized animal tissues, such as the liver, are desired.

### SUMMARY OF THE INVENTION

A regenerative tissue repair product may comprise a decellularized extracellular matrix derived from animal liver. The decellularized extracellular matrix may retain the collagen morphology of the animal liver prior to decellularization and may have less than 1,000 ng, and in an example, less than 500 ng, DNA/mg dry extracellular matrix and greater than 760 μg total collagen per milligram dry extracellular matrix, the collagen comprising soluble collagen and insoluble collagen.

The decellularized extracellular matrix may have greater than 1.6 μg glycosaminoglycans per milligram of dry extracellular matrix and greater than 150 μg elastin per milligram of dry extracellular matrix. The decellularized extracellular matrix may be configured into a single or multi-layer sheet or powder. The sheet formed from the decellularized extracellular matrix may be about 0.5 mm to 3.2 mm thick.

The decellularized extracellular matrix may retain the natural reticular, honeycomb morphology of the animal liver prior to decellularization. The decellularized extracellular matrix may retain substantially the amount of collagen and proteins associated with the extracellular matrix before decellularization to recruit stem cells and promote optimal cellular growth in surrounding tissues when applied onto tissue to aid in healing tissue to be repaired. The animal liver may comprise a porcine liver. The decellularized extracellular matrix may have greater than 150 μg elastin per milligram dry extracellular matrix and greater than 110 mg/ml of protein.

Another aspect is directed to a method of forming a regenerative tissue repair product. The method may comprise slicing an animal liver into a liver slice. The method may further include pressing and rinsing the liver slice to form a decellularized extracellular matrix from the liver slice that retains the collagen morphology of the animal liver prior to decellularization and has less than 1,000 ng DNA/mg dry extracellular matrix and greater than 760 μg total collagen per milligram dry extracellular matrix comprising soluble collagen and insoluble collagen.

### BRIEF DESCRIPTION OF THE DRAWINGS

Other objects, features and advantages of the present invention will become apparent from the Detailed Description of the invention which follows, when considered in light of the accompanying drawings in which:

FIG. 1 is a high-level flowchart showing a basic sequence of manufacturing the regenerative tissue repair product according to the invention.

FIG. 2A is a block diagram of the slicer used for slicing the liver.

FIG. 2B is a block diagram of the indicator scale and precise scale that measures and weighs liver slices.

FIG. 3 is an image of an Eberbach shaker table holding jars containing liver slices.

FIG. 4 is an image of a mechanical press to press liver slices and capture fluid.

FIG. 5 is a block diagram showing the detergent solution components.

FIG. 6 is another image of an Eberbach shaker table holding jars containing liver slices after initial pressing.

FIG. 7 is a bar chart showing the DNA quantification of the regenerative tissue repair product compared with a commercially available product.

FIG. 8 is a bar chart showing the DNA quantification by lot of different samples of the regenerative tissue repair product compared with a commercially available product.

FIG. 9 is a bar chart showing the total collagen quantification of the regenerative tissue repair product compared with a commercially available product.

FIG. 10 is a bar chart showing the total collagen quantification by lot of different samples of the regenerative tissue repair product compared with a commercially available product.

FIG. 11 is a bar chart showing the level of sulfated glycosaminoglycans (sGAG) of the regenerative tissue repair product compared with a commercially available product.

FIG. 12 is a bar chart showing the elastin quantification of the regenerative tissue repair product compared with a commercially available product.

FIG. 13 is a bar chart showing that the percent residual moisture content of the regenerative tissue repair product was maintained during storage (shelf life).

FIG. 14 is a bar chart showing that the mean onset glass transition temperature (Tg) of the dry regenerative tissue repair product was maintained during storage (shelf life) demonstrating device stability.

FIG. 15 are images of the regenerative tissue repair product and competitor product showing identification of mature (Type I) collagen fibers (shown by the pattern section) and immature (Type III) collagen fibers (shown by the solid section).

FIG. 16 is a chart showing locations in the press that have forces measured during the liver slice pressing.

FIG. 17 is a chart showing the press force in pounds for each location when pressing different samples.

FIG. 18 is a high-level flowchart showing an example process for manufacturing the regenerative tissue repair product according to the invention.

FIG. 19 is an image of a tissue repair product showing fractured collagen when no control is exerted over the liver slice pressing.

FIG. 20 is an image of the regenerative tissue repair product according to the invention showing the improved honeycomb shape and consistency of collagen when there is control over the liver slice pressing.

### DETAILED DESCRIPTION

Different embodiments will now be described more fully hereinafter with reference to the accompanying drawings, in which preferred embodiments are shown. Many different forms can be set forth and described embodiments should not be construed as limited to the embodiments set forth herein. Rather, these embodiments are provided so that this disclosure will be thorough and complete, and will fully convey the scope to those skilled in the art.

The tissue material is a regenerative tissue repair product that may be used for skin grafting and other wound care and similar applications. The product is derived from the extracellular matrix (ECM) of decellularized porcine liver or other animal source. The liver has been processed to remove its cells and maintain its naturally occurring bioactive components, such as proteins, collagen, growth factors, proteoglycans, glycoproteins, and other similar non-DNA and noncellular components.

The regenerative tissue repair product is preferably formed in a sheet form and may be produced by solubilizing and reforming the ECM product into a single or multi-layer sheet that is about 0.5 mm to about 3.2 mm thick in a non-limiting example. It may also be provided in other forms such as powder and flakes, hydrogel, surgical sheets and multi-layer surgical sheets. The regenerative tissue repair product is beneficial in helping heal a wide variety of wounds or damaged tissue by delivering bioactive components to the injury or surgical site. The powder or hydrogel could be added to the sheet or multi-layer sheet. The powder or hydrogel would degrade faster than the sheet enabling the bioactive molecules that promote cell proliferation and tissue regeneration to occur faster than use of the sheet alone. An extract from the liver may be combined with the material as the regenerative tissue repair product formed from the extracellular matrix of the decellularized animal liver to enhance the native bioactive components. Liver extracts are rich in growth factors such as Hepatocyte Growth Factor (HGF) and Transforming Growth Factor-beta (TGF-β), which are crucial for tissue repair and regeneration. These extracts can be incorporated into the sheet or hydrogel forms to promote cellular activities essential for healing.

Because it is difficult to remove the cells from the liver ECM using typical chemicals associated with the decellularization processes, the liver's natural honeycomb collagen structure maintains hold of the cells during decellularization. Increasing the amount of chemicals and their application time to decellularize the liver may ultimately remove most of the proteins, reducing the beneficial effect of the final produced regenerative tissue repair product.

In accordance with a non-limiting example, it has been found that force may be applied to the liver extracellular matrix (LEM) at different amounts prior to and after chemical processing used in decellularization, and prior to a final chemical process, to preserve the collagen architecture and morphology of the extracellular matrix that exists prior to decellularization, and thus, maintain the maximum number of proteins in this final product. The specific forces required at each mechanical process to aid in removing the cells prevent damage to the extracellular matrix and maintain the natural liver morphology. It is possible to use a mechanical or pneumatic press with a range in force varying at each process step to maintain the natural liver morphology architecture and proteins. The extracellular matrix derived from the liver may be a different thickness at each of the processing steps, making it necessary to tailor the amount of applied force to maximize the removal of unwanted cellular material. The final product as a sheet for the regenerative tissue repair product has a lower DNA content, while maintaining total collagen, glycosaminoglycans (GAGs), proteins and other non-cellular components.

In accordance with a non-limiting example, because it is difficult for chemicals alone to remove the cells from the liver honeycomb structure, a pressing force, such as from a mechanical or pneumatic press, is added to the process steps to remove cells without additional chemicals. For example, a first pressing force against the liver sample may be added prior to the addition of a first chemical mixture in order to compress fluid out of the cells and aid in pulling the chemical into the cells. A second pressing force may be applied after the first applied chemical mixture, and a third pressing force may be applied prior to application of a second chemical mixture. These pressing forces compress fluid out of the cells to help pull the chemicals into the cells.

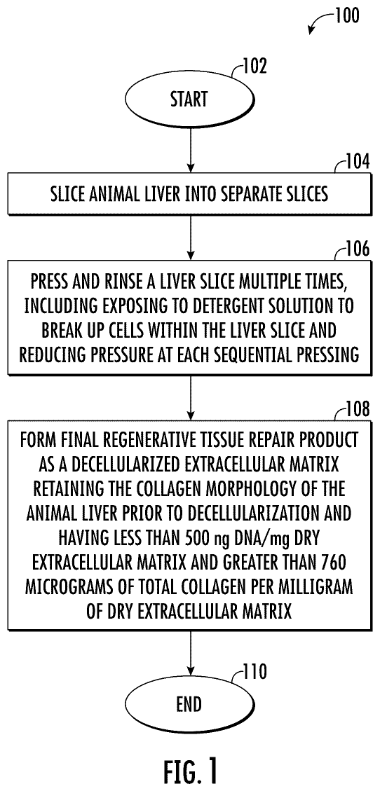

Referring now to FIG. 1, a high-level sequence diagram shown generally at 100 illustrates a procedure for making the regenerative tissue repair product derived from extracellular matrix of decellularized animal liver, such as porcine liver. The process starts (Block 102) by first slicing an animal liver into at least one liver slice, such as a plurality of separate slices (Block 104). A liver slice is pressed and rinsed, which includes exposing to a detergent solution as explained below to break-up cells within the liver slice. The pressure may be reduced in each sequential pressing (Block 106). The final regenerative tissue repair product is formed as a decellularized extracellular matrix retaining the collagen morphology of the animal liver prior to decellularization and having less than 1,000 ng, and in an example, less than 500 ng DNA/mg dry extracellular matrix and greater than 760 microgram total collagen per milligram of dry extracellular matrix comprising soluble and insoluble collagen (Block 108). The process ends (Block 110).

The process includes pressing and rinsing the liver slice multiple times such as pressing the liver slice a first time, exposing the pressed liver slice to a detergent solution to aid in breaking up the cells within the liver slice, and pressing the liver slice a second time. The first pressing may occur at a higher pressure than the second pressing, and at a time period longer than the second pressing. The liver slice may be pressed a third time at a pressure lower than the pressures of the first and second pressings. As explained in detail below, the press may have a mesh configured to allow fluid and cellular debris to move away from the liver slice. The liver slice may be exposed to a higher pH rinsing after the third pressing to facilitate an osmolality change to aid in removing cellular debris.

An example procedure is now explained in greater detail below. This example procedure may start with receiving purchased livers, followed by producing porcine liver slices, and subsequently decellularizing the liver slices. The decellularized liver slices may become a base material for multiple final products, including a wound care sheet that operates as a regenerative tissue repair product and may be applied onto partial or full thickness wounds, pressure, diabetic, venous and chronic vascular ulcers, second degree burns, surgical wounds, such as donor sites/grafts, post-Moh's surgical wounds, post-laser surgical wounds, podiatric, trauma wounds, abrasions, lacerations, and tunneled/undermined wounds.

In this example, livers were received from a supplier or other source in a fresh condition and in cold shippers. The livers were subsequently washed and inspected. The liver lobes were separated and placed into a −40 Degrees Centigrade freezer, preferably on trays, to provide a flat surface for freezing. It was found that freezing the livers with a flat side facilitated liver slicing.

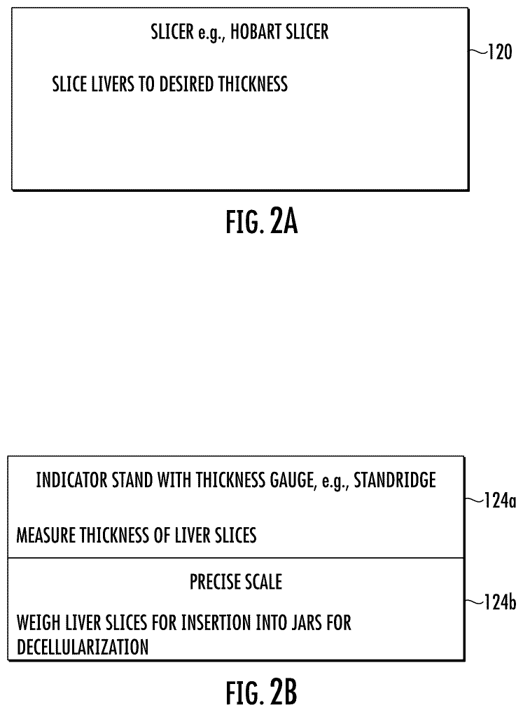

The livers were removed from the freezer and each lobe was thawed at room temperature for approximately 30 minutes. The thaw time may vary depending on the size and thickness of individual liver lobes. Partially thawed livers were sliced on a Hobart Slicer, such as shown at 120 of FIG. 2A, to produce the required thickness, which was monitored using a thickness gauge, for example, a standridge indicator stand with a thickness gauge (FIG. 2B) shown at 124a. The thickness of the slice was controlled for the decellularization process. In a non-limiting example, the slice thicknesses were between about 0.5 mm to 3.2 mm, and in a working example, about 2.5 mm to about 3.2 mm. The decellularization process as described produces the final product from this initial thickness range. It is possible that thicker liver slices may be processed and the decellularization process modified for these thicker liver slices.

In the example for processing the liver slices, they are weighed on a precise scale 124b and placed into glass jars for future decellularization. An accurate weight is used to maintain control of the amount of liver within the jar versus the washing materials used during decellularization. In this working example, the weight of liver slices collected per jar for this slice thickness was about 300 grams.

The decellularization process proceeds. The liver slices are decellularized using a series of washes that remove the cellular content from the liver slices. The washing includes use of deionized water to rinse and wash away chemicals used during the process, and remove the cellular debris created by the chemicals. A primary detergent solution may use a mixture of chemicals that breakup the cells within the liver slices. The cellular debris created during this processing step was removed manually in this example, but more automatic, pneumatically controlled presses may be used. The liver slices are pressed, which slides and pushes out the cellular debris. Additional washes with deionized water and chemical treatment further clean the liver slices to produce the decellularized liver slices. Upon completion of this process, the liver slices are processed to produce final regenerative tissue repair product as derived from the extracellular matrix of the decellularized animal liver.

Many tissue repair products currently on the market are processed using a chemical only or chemical and/or sonication and/or agitation decellularization process. Differences between these decellularization processes exist, notably in the type of chemicals, the amount of chemicals, and the time the chemicals are applied. Deionized water is typically used as a rinse in these processes.

As noted before, the honeycomb structure of the liver collagen makes it difficult to decellularize the liver and higher concentrations of chemicals must be used and/or longer time periods in the chemical application to decellularize the liver. Nonionic detergents, such as Triton, have typically been used and applied for about 24 to 48 hours. The commercially available ECM product produced via chemical only processing as a tissue or wound treatment from the previous commercial techniques was inadequate because the final product included a high amount of remaining DNA, which causes an inflammatory response upon implantation. If enough DNA had been removed, then the liver was damaged by the Triton, which left minimal proteins and a denatured collagen. As a result, the final tissue product produced from these processes was limited in its capacity to regenerate new tissue during implantation.

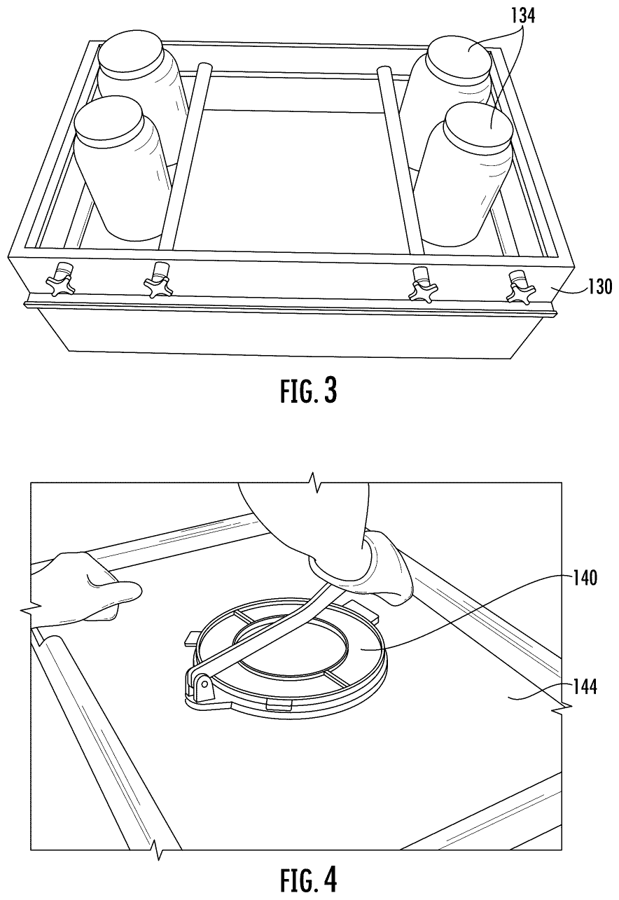

In the process of the current invention, deionized water was prepared for the rinse procedure. Four-liter jars were employed in this process. For each jar of 300 grams of liver slices to be decellularized, the 4-liter jar was filled with two liters of deionized water. In an example process, three rinses with deionized water were accomplished. Liver slices were placed into prepared 4-liter jars with deionized water. The 4-liter jars were placed on an Eberbach shaker, which was programmed using the program setpoints: 1) time: 15 minutes; and 2) speed: 178 RPM. The 4-liter jars were removed from the shaker table and the contents poured through a strainer held over a sink. The 4-liter jars were rinsed with deionized water to ensure no tissue or cellular content remained in the jar. The 4-liter jars were filled with two liters of deionized water. Liver slices were poured from the strainer into a 4-liter jar and the lid placed on the jar. The deionized water first rinse was repeated for a total of three rinses. These initial deionized water washes, as rinses helped remove red blood cells and some cellular debris from the slices. An example for the shaker table 130 and the jars 134 holding liver slices is shown in FIG. 3.

The process continued with use of the first press. The press station included, in this example, a mechanical press and polypropylene mesh having an opening size of 0.05″×0.035″.The mechanical press was placed on a tray with raised edges to capture fluid. The liver slices were removed from the strainer and placed on the mesh in the mechanical press to allow fluid/cellular debris to move away from liver slice and into trays during pressing. The press was closed onto the slices firmly, while applying constant pressure at approximately 0.7 to 0.8 psi for 15 to 30 seconds. This initial pressing removed fluid/cellular debris and prepared the pressed liver to draw the Triton Detergent Solution into the liver slice. An example of the mechanical press 140 and its press station 144 is shown in FIG. 4.

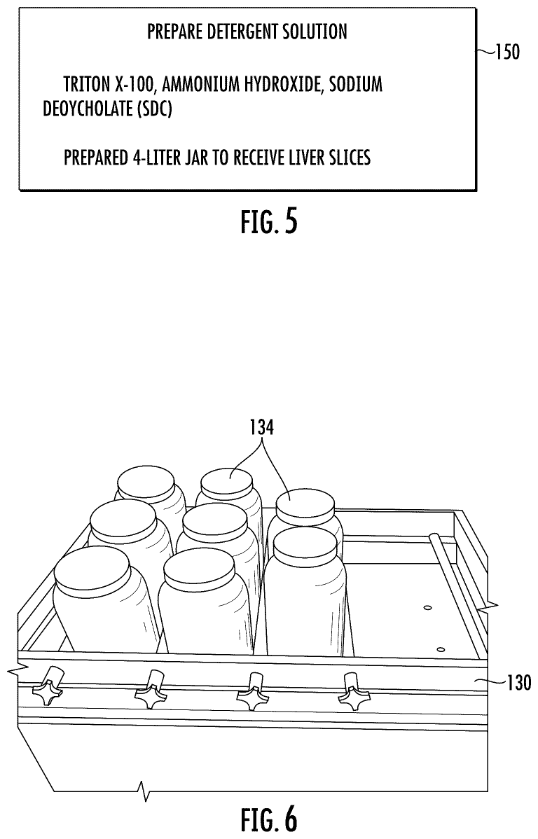

A Triton detergent solution was prepared by initially placing Triton X-100, also referred to as octylphenol ethylene oxide condensate, ammonium hydroxide, SDC (sodium deoxycholate detergent), a 4-liter glass jar, a scale, a weigh dish, and two pipettes on a stainless steel table. For each 300 grams of liver slices to be decellularized, one (1) 4-liter jar of detergent solution was prepared. Two liters of deionized water were added to each 4-liter jar. About 25 ml±1 ml of Triton X-100 was measured and added to each 4-liter jar. About 2.5 ml±0.1 ml of ammonium hydroxide was measured into each 4-liter jar. About 5 grams of SDC was weighed on a scale and then added to each 4-liter jar, and a lid placed on the jars. Each 4-liter jar was placed onto a shaker and the detergent solution mixed by shaking at 178 RPM for 15 minutes. An example of this preparation is shown in the block diagram at 150 of FIG. 5.

The Triton detergent wash procedure is now explained. After the first mechanical pressing, the liver slices were placed into a prepared 4-liter jar with the detergent solution. The pressed lives slices draw in and absorb the Triton detergent solution. The 4-liter jars were placed on the Eberbach shaker (FIG. 6) and the liver slices washed for about 60 minutes at 178 RPM. The 4-liter jars were removed and contents poured through a strainer in preparation for the second mechanical pressing.

This Triton wash adds a pH change, and a primary purpose is to disrupt cell membranes and aid the removal of cellular debris. By adding in the mechanical press cycle, the process reduces the typical concentration and wash time of the Triton solution, thus preserving collagen, proteins and growth factors. The secondary purpose of the wash using the Triton solution is to remove any viruses that may be within the tissue.

The second cycle for mechanical pressing started. Liver slices were removed from the strainer and placed on the mesh in the mechanical press. The press was closed onto the slices firmly, while applying constant pressure, e.g., about 0.6 to 0.7 psi, for about 10 to 30 seconds. After this second pressing, the liver slices were placed into a 4-liter jar with two liters of deionized water to begin the next washing steps. This second pressing disrupted cellular membranes, and was the primary pressing to remove cellular debris from the liver slices that were disrupted during the previous Triton detergent wash step.

A deionized water rinse of 4-6 cycles was next accomplished. Liver slices were placed into prepared 4-liter jars with deionized water. The 4-liter jars were placed on the Eberbach shaker and liver slices washed for about 15 minutes at 178 RPM. The 4-liter jars were removed from the shaker table and contents poured through a strainer. Liver slices were placed into new 4-liter jars with deionized water. The deionized water rinsing occurs for a total of three rinses, which removed the cellular debris from the liver slices, facilitated by the Triton and mechanical pressing.

A third mechanical pressing cycle was performed. The liver slices were removed from the strainer after the deionized water rinses and placed on the mesh in the mechanical press. The press was closed onto the liver slices firmly, while applying constant pressure, e.g., about 0.5 to 0.6 psi, for about 10 to 15 seconds. After pressing, the liver slices were placed into a 4-liter jar with a prepared NaOH solution. This pressing step helped drive fluid from the liver to aid in drawing the NaOH used in the next step into the liver slices and disrupt any remaining cellular membranes.

The sodium hydroxide (NaOH) solution was prepared. The NaOH, a 4-liter glass jar, and scale were placed on a stainless steel table. For each 300 grams of liver slices to be decellularized, one (1) 4-liter jar of NaOH solution was prepared. The process continued and two liters of deionized water were added to each 4-liter jar. Eight (8) +0.1 grams of NaOH were added to each 4-liter jar. Each 4-liter jar was placed onto the shaker table, and the NaOH solution mixed by shaking at 178 RPM for 5 minutes.

The sodium hydroxide wash procedure was next. After the third mechanical pressing cycle was complete, liver slices were placed into the jars of the prepared NaOH solution. Each 4-liter jar was placed onto the shaker table and the liver slices were washed for about 30 minutes by shaking at 178 RPM. This NaOH solution had a high pH, which facilitated an osmolality change to help remove remaining cellular debris. It also helped reduce the sugar molecular alpha-gal remaining in the material. The liver slices were at their weakest state after the NaOH wash and can easily be torn. After the NaOH washing was complete, the contents were poured through a strainer and into the 4-liter jar with two liters of deionized water for a deionized water rinse (7-9).

In this rinsing (7-9), liver slices were placed into a prepared 4-liter jar with deionized water. The 4-liter jars were placed on the Eberbach shaker and liver slices were washed for about 15 minutes at 178 RPM. The 4-liter jars were removed from the shaker table and the contents poured through a strainer. Liver slices were placed into new 4-liter jars with deionized water and the deionized rinse was repeated for a total of three rinses. These last deionized water washes removed the remaining cellular debris and any remaining chemicals from the liver slices. These washes also brought the liver slices back to nominal pH and the honeycomb collagen structure of the liver strength returned.

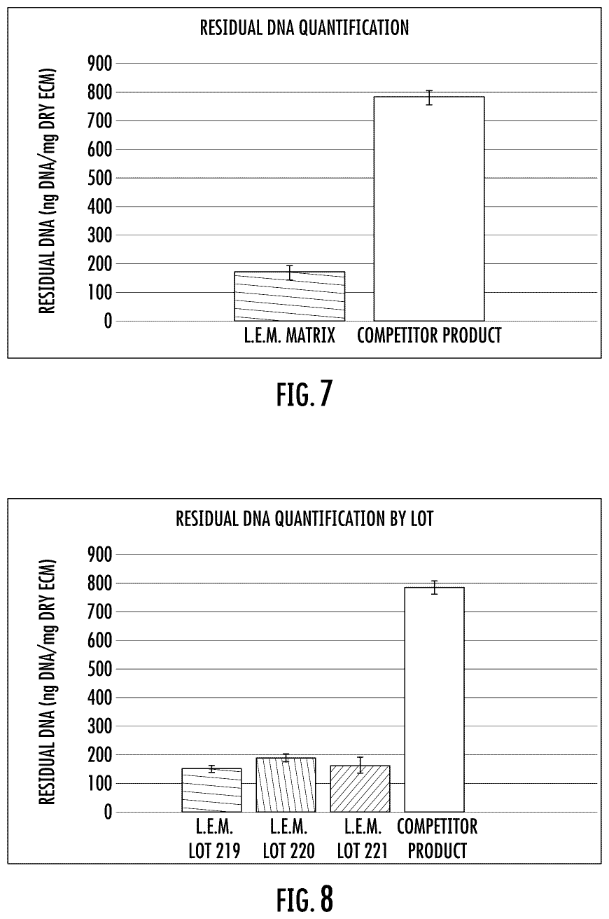

After the last deionized water rinse, the 4-liter jars were placed in the refrigerator to prepare for lyophilization. After lyophilization, the wound sheets as the regenerative tissue repair product derived from the extracellular matrix of the decellularized animal liver were cut to several different sizes, packaged, and shipped out for electron-beam (e-beam) sterilization. This process as described was performed for several product runs, some of the results shown and described with reference to the charts and tables of FIGS. 7-11.

The final regenerative tissue materials as a wound and regenerative tissue repair product are now described. The regenerative tissue repair product, in accordance with the described embodiment, is used for the management of wounds, including partial and full thickness wounds, pressure ulcers, diabetic ulcers, venous ulcers, chronic vascular ulcers, tunneled/undermined wounds, surgical wounds, trauma wounds, and draining wounds, commonly referred to as soft tissue repair. This regenerative tissue repair product facilitates the repair and regeneration of damaged tissue by inducing the body's own regenerative capabilities. The regenerative tissue repair product works by recruiting stem cells to the implant site, modulating the body's inflammatory response to that of a regenerative response, and inducing angiogenesis to form new blood vessels.

The regenerative tissue repair product is

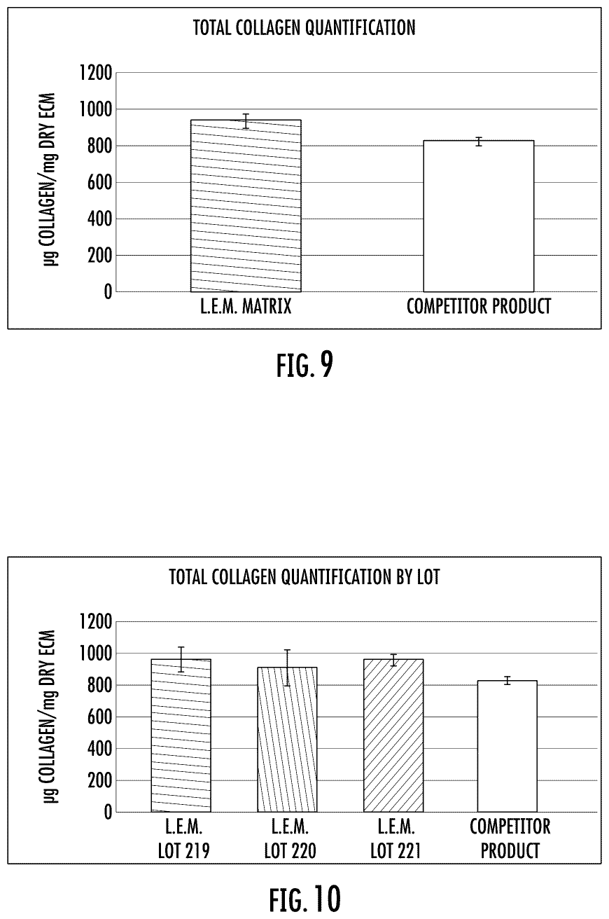

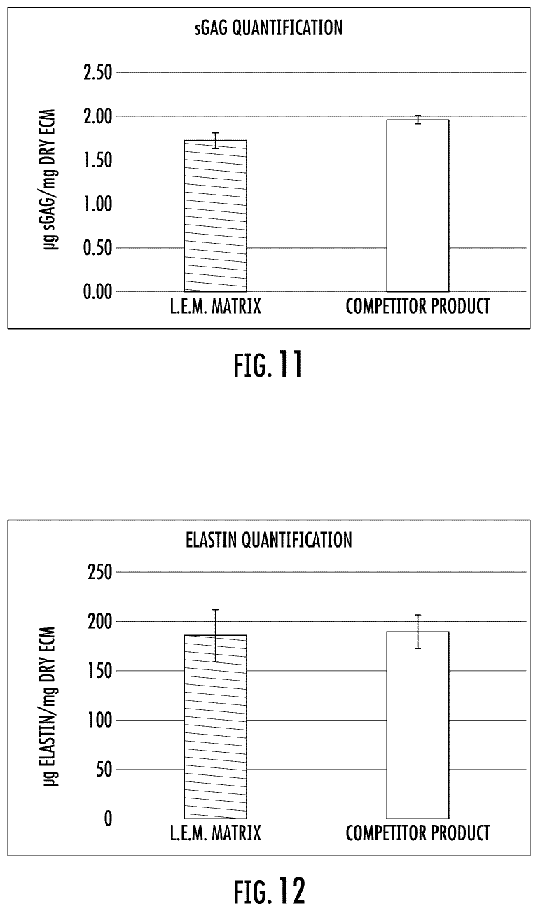

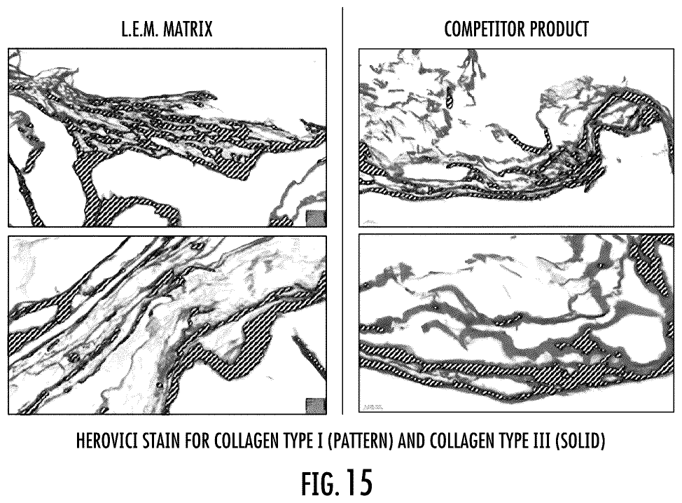

predominately collagen, and includes additional components such as elastin, glycosaminoglycans, fibronectin, and laminin. The amount of DNA, collagen, glycosaminoglycans (GAGs), and elastin in the regenerative tissue material as the ECM derived from the decellularized liver has been compared with a competitive product and the bar charts and images for these results are shown in FIGS. 7-15. DNA content is known to cause an inflammatory reaction when it remains in the implanted regenerative tissue ECM material. The testing indicates a very low level of DNA compared to other commercially available products (FIGS. 7-8). The testing demonstrates the product is primarily collagen based (FIGS. 9-10). The regenerative tissue product has slightly greater total collagen as compared to a commercially available product. The collagens are primarily Type I collagens (shown by the pattern section) and Type III collagens (shown by the solid section) (FIG. 15). The GAGs are attached to proteins and the GAG amounts in the regenerative tissue material are similar to a commercially available product (FIG. 11). The elastin content of the regenerative tissue product is comparable to a commercially available product. Further development may include testing for other collagen types and protein, including individual protein markers.

Referring now to the bar chart shown in FIG. 8, the DNA quantification by lot is illustrated for the regenerative tissue repair product as compared to another commercially available product. The DNA data by lot for the regenerative tissue repair product is listed as L.E.M. Lot 219 through L.E.M. Lot 221. The average of the runs is shown in the bar chart of FIG. 7 where the LEM (liver extracellular matrix) wound sheet corresponds to the regenerative tissue repair product. The removal of DNA has improved and been refined in the process.

It is known that the extracellular matrix devices that contain significantly more cellular material, such as the competitor product, as compared to the regenerative tissue repair product will show a predominantly M1 pro-inflammatory macrophage response and subsequent fibrosis, while those tissue repair products that contain less DNA would demonstrate a predominantly M2 wound healing/anti-inflammatory macrophage response.

It is known that macrophages perform different functions in homeostatic and immune responses and are highly specialized in sensing the microenvironment and modify their properties accordingly. Macrophages are often categorized as pro-inflammatory (M1) and anti-inflammatory/pro-resolving (M2) profiles.

The M1 macrophages start and sustain inflammatory responses, secrete pro-inflammatory cytokines, activate endothelial cells, and induce the recruitment of other immune cells into the inflamed tissue. M2 macrophages promote the resolution of inflammation, phagocytose apoptotic cells, drive collagen deposition, coordinate tissue integrity, and release anti-inflammatory mediators with cell metabolism switching macrophage phenotypic and functional changes. For example, M1 macrophages employ glycolysis and present two breaks on the TCA cycle that result in accumulation of itaconate (a microbicide compound) and succinate, which may induce Hypoxia Inducible Factor 1α (HIF1α) stabilization. There may be transcription of glycolytic genes and glycolytic metabolism of M1 macrophages.

The M2 macrophages depend on oxidative phosphorylation (OXPHOS). The TCA cycle helps induce the electron transport chain (ETC) and regulate the metabolism of lipids and amino acids and affect their responses.

The mechanical pressing as described removes substantial amounts of DNA as indicated in the results and maintains the high levels of proteins existing in the tissue repair product. The tissue repair and wound care product as the regenerative tissue repair product may be formed as a thin sheet ranging in thickness from about 0.5 mm to about 3.2 mm. It is also possible to manufacture from the described process a powder version. It is also possible to manufacture the product as a single or multi-layer sheet. A bacterial coating may be formed on the sheet or multiple sheets when a multi-layer sheet is formed and aid in reducing the chance of infection.

It is also possible to produce stacked or multiple layers. Multiple layers of the regenerative tissue repair product may be stacked and placed into a lyophilizer or vacuum oven. The vacuum of the lyophilizer or vacuum oven pulls and holds the collagen together during the process, and the multiple layers remain as a single thicker regenerative tissue material after lyophilization. This stacked product as a regenerative tissue repair product may be used for the same wound indications, and a surgical application that requires additional strength, e.g., a hernia repair. Another possible regenerative tissue repair product may begin with an initial thicker slice, which requires some modifications to the decellularization and mechanical press process.

The liver honeycomb structure is important for making the regenerative tissue repair product. The liver collagen matrix refers to the network of collagen fibers that make up the connective tissue structure of the liver. The collagen is the protein that provides strength and support to the tissues in the body. The liver collagen matrix primarily includes Type I collagen, and forms thick, strong fibers that provide tensile strength to the liver tissue. These collagen fibers are arranged in a reticular honeycomb pattern, creating a three-dimensional network throughout the liver. It surrounds the blood vessels and bile ducts within the liver, providing structural support and helping to maintain the shape and integrity of these structures and for the final regenerative tissue material (FIG. 15). Type III collagen is often found alongside Type I collagen. The Type III fibers are thinner, imparting elasticity and creating a supportive structure in soft tissues (FIG. 15). This ECM matrix in the regenerative tissue repair product provides a scaffold for the attachment of other cells and molecules involved in regeneration and repair of the tissue for wound care.

Microscopically, the collagen matrix appears as fibers that crisscross throughout the liver tissue. The collagen fibers can be seen branching and interweaving with each other, forming a complex network, which helps stabilize and reinforce the liver tissue, preventing it from collapsing or distorting under mechanical stress. The liver collagen matrix is important to the final regenerative tissue repair product since it provides strength and support, while also contributing to its function and regenerative capacity.

The organs that are best known for promoting cellular growth and tissue repair are the liver, small intestine, and bladder. The liver, however, is the only organ that can regenerate readily. For that reason, with its honeycomb structure, it is important in producing regenerative tissue material.

Liver contains Type I, Type III, and basement membrane Type IV collagens. There are also several proteins that are unique to the liver and are not found in all organs. The following examples explain five of these proteins.

1. Albumin: This is the most abundant protein in the blood and is exclusively synthesized by liver cells. It plays a role in maintaining blood pressure and transporting various substances in the blood.

2. Fibrinogen: This protein is involved in blood clotting and is produced by liver cells. It is converted into fibrin during the clotting process.

3. Alpha-1-antitrypsin: This protein is produced in the liver and helps protect the lungs from damage caused by enzymes released during inflammation.

4. Transferrin: This protein plays a role in the transport of iron in the blood and is primarily synthesized in the liver.

5. IGF-1 (Insulin-like growth factor 1): This protein is involved in cell growth and the regulation of metabolism. It is primarily produced in the liver.

These example proteins are synthesized in the liver and not found in all organs. The liver has a unique role in protein synthesis and metabolism, which contributes to its diverse and vital functions in the body.

The liver also plays a crucial role in cellular growth. It synthesizes and releases IGF-1, which is a hormone that plays a key role in cell growth. It also promotes cell proliferation, DNA synthesis, and tissue growth. The liver produces and releases other growth factors (growth hormone and albumin), hormones, and proteins that regulate growth and development. Some of the key growth factors in the liver include:

1. Hepatocyte growth factor (HGF): HGF is a potent mitogen that stimulates the growth and division of hepatocytes, the main functional cells of the liver. It is secreted by various cell types in the liver, including hepatic stellate cells, and plays a critical role in liver regeneration after injury.

2. Epidermal growth factor (EGF): EGF is a growth factor that stimulates the growth and proliferation of various cell types, including hepatocytes. It is produced by hepatocytes and acts in an autocrine manner to promote liver regeneration.

3. Transforming growth factor alpha (TGF-α): TGF-α is a growth factor that is structurally similar to EGF and acts through the same receptor. It is produced by hepatocytes and acts in a paracrine manner to stimulate the growth and division of nearby hepatocytes during liver regeneration.

4. Fibroblast growth factor (FGF): FGFs are a family of growth factors that play important roles in tissue development and repair. In the liver, FGFs stimulate hepatocyte proliferation and promote liver regeneration.

5. Vascular endothelial growth factor (VEGF): VEGF is a growth factor that stimulates the growth of blood vessels. In the liver, VEGF plays a crucial role in angiogenesis, the process of forming new blood vessels, which is essential for liver regeneration and tissue repair.

The liver contributes to cellular growth through the production and release of growth factors, hormones, and proteins that regulate cell proliferation, DNA synthesis, and tissue growth. The regenerative tissue repair product derived from the extracellular matrix of the decellularized liver is manufactured to maintain its collagen and proteins and provide the necessary growth factors, hormones, proteins, and recruited stem cells to promote optimal cellular growth in the surrounding, healing tissue.

Referring now again to FIG. 11, a bar chart of the amount of tested glycosaminoglycans (GAGs) is illustrated, where the regenerative tissue repair product derived from the extracellular matrix of the decellularized porcine liver is compared to competitor product. The testing indicates the liver derived regenerative tissue repair product maintains a reduced level of GAGs as compared to the other commercially available product. In this example, the glycosaminoglycans (GAGs) are sulfated glycosaminoglycans as linear polysaccharides that are usually negatively charged. They may include repeating disaccharide units that contain uronic acid and N-acetylhexosamine. Some may have different repeating sub-units.

GAGs play an important role in an extracellular matrix (ECM) in the current liver derived regenerative tissue repair product, and also indicate a strong protein presence in the material. The glycosaminoglycans are important components of the extracellular matrix (ECM) because they help maintain its structure and function. GAGs are long, unbranched polysaccharide chains that are composed of repeating disaccharide units. They are negatively charged due to the presence of sulfate or carboxylate groups, which allows them to interact with other components of the ECM, such as proteins and water.

One of the main functions of GAGs is to provide hydration to the ECM. Due to their negative charge, GAGs attract and bind to water molecules, creating a hydrated gel-like network. This hydration is important for maintaining the proper mechanical properties of the ECM, as well as facilitating the diffusion of nutrients and waste products.

GAGs also play a role in cell adhesion and migration. They can bind to proteins, such as fibronectin and collagen, forming attachments between cells and the ECM. This interaction is important for cell signaling and provides a scaffold for cell migration during tissue repair and development. GAGs can also sequester growth factors and other signaling molecules, preventing their degradation and prolonging their activity. This allows for the regulation of various cellular processes, including tissue development, wound healing, and immune response.

Overall, GAGs are crucial for maintaining the structure, function, and integrity of the ECM. They provide hydration, facilitate cell adhesion and migration, and regulate cell signaling. Without GAGs, the ECM would not be able to properly support and regulate the activities of cells within tissues and organs.

Elastin is a highly stable protein of the extracellular matrix. Elastin imparts elasticity to adult tissue and comprises the major component in elastic fibers. Tissue processing steps, such as decellularization, may affect the content and quality of elastin. The regenerative tissue repair product retained a comparable level of elastin as compared to the competitor product (FIG. 12).

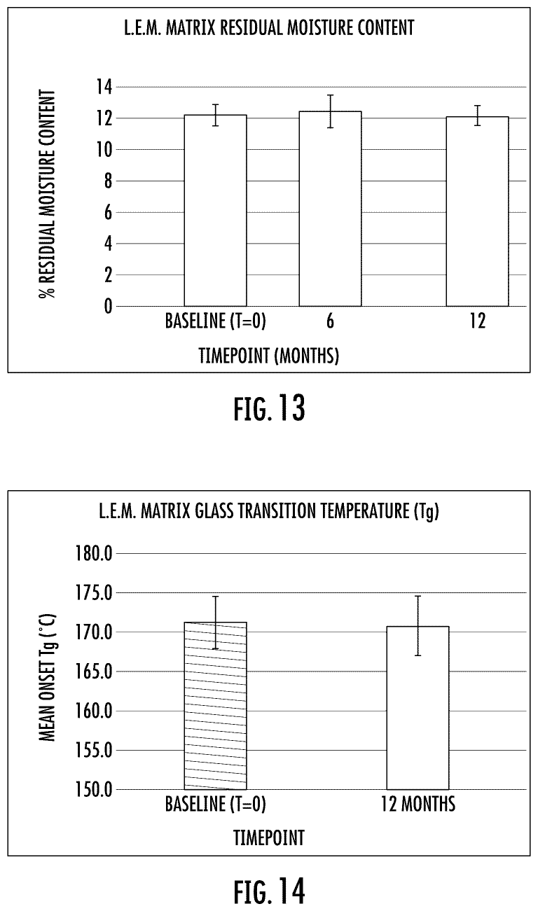

The regenerative tissue repair product is beneficial because it maintains its residual moisture content during storage (shelf life) as illustrated in the bar chart of FIG. 13, showing the different levels that are substantially the same at the baseline, six months, and twelve months. Also, the mean onset glass transition temperature (Tg) of the regenerative tissue repair product was maintained during shelf life and storage as shown in the bar chart of FIG. 14.

Initial testing and processing to produce the regenerative tissue repair product as described above used mechanical presses during the decellularization process to determine optimal machine pressures and press time duration on different liver slice samples to obtain the highest quality results. Different mechanical press specifications that were required to expel fluid from the porcine liver tissue slices were tested. Two different presses were tested.

The first press was an 8-inch tortilla press with a painted aluminum press surface carrying a 1 millimeter thick polypropylene mesh to aid in expelling fluid from the liver slices. The second press was a 16 inch by 16 inch manual dough press with an aluminum press surface carrying the polypropylene mesh. Both presses included a FlexiForce compression sensor, such as a FlexiForce B201 sensor, that measured the compression force between two surfaces. The sensor was calibrated with weights of 2.5 pounds, 5 pounds, and 10 pounds. The calibration adjusted sensitivity and maximum load readouts. Inputting 10 pounds in the FlexiForce software as 5% of the potential maximum load of the press permitted the visual force output of 200 pounds.

Each of the first and second presses employed different testing techniques. With the first tortilla press, the force testing was done using a standard scale with an operator simulating the force required to press slices during manufacturing. An estimated 25 to 35 pounds of force was applied manually for 5 seconds per slice. Testing with shims ranged from 0.1 millimeter at 0.01 millimeter intervals to determine if the fixed dimension depth versus the pressure applied resulted in cleaner liver slices during the decellularization process. The shims were placed on the mesh, next to the porcine liver tissue during the pressing stages of the decellularization process. They served as a fixed dimension for the operator to press, instead of relying on the estimated weight applied to the press by hand. Pressing down more to create thinner slices yielded cleaner slices on average. The lower that operators pressed, the more fluid and cellular content were expelled from the porcine liver tissue.

The manual dough press was also tested. Some alterations were made to that press. The aluminum knobs present at the four corners of the top platen that operated as a fixed stop were removed. The press was disassembled, leveled and reassembled to allow for more even distribution of the forces across the surface of the platen.

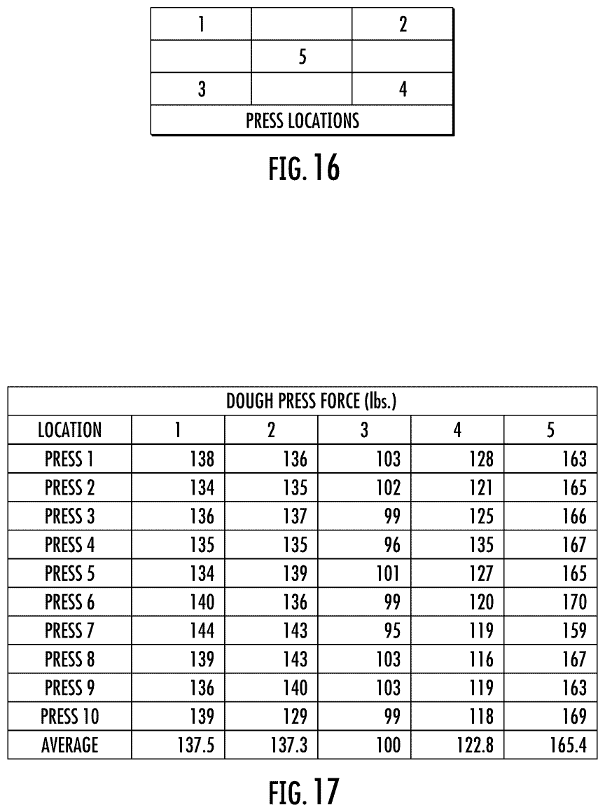

About nine slices of porcine liver tissue were placed onto the platen surface of the second dough press having the mesh to aid in expelling fluid. The FlexiForce compression sensors determined the force applied to the liver slices across the platen. In an experiment, a sensor was placed directly on the liver slices to measure the force received on the porcine liver tissue during pressing. The sensor positioned on slices proved inconsistent. The measurements could not be read on the software, and thus, the porcine liver tissue was unstable, preventing the sensor from obtaining stable measurements. Five locations were tested across the platen surface of the dough press as shown in FIG. 16, and the force measurements taking 10 pounds per location with the results shown in the chart of FIG. 17.

The first tortilla press demonstrated that adequate pressure may be applied to the porcine liver tissue to accomplish the production of the regenerative tissue repair product. The average applied force was 30 pounds, and the pressure output of 0.6 psi (force applied/working area) was enough to expel fluid from the porcine liver tissues. Acceptable liver tissue slices were white to off-white or included light yellow/brown areas, while rejected liver slices included dark brown areas. The pressed porcine liver tissue slices had clear, but minor light brown spots. The press proved to remove cellular content according to the visual criteria.

The second dough press improved the overall working and the force applied when decreased in pressure output. The dough press averaged 132.6 pounds across 256 square inches, or about a pressure output of about 0.52 psi. Force measurements taken across the five locations of the platen, as shown in the layout of FIG. 17, showed the uneven distribution of pressure. Location 3 as shown averaged the lowest at 100 pounds of force applied, while location 5 averaged the highest at 165.4 pounds of force. A visual inspection saw there were inconsistencies in pressure across the porcine liver tissue and brown spots could be seen in areas of lower psi at locations 3 and 4. The porcine liver tissue that was placed in areas of higher force remained visibly clear.

These results evidence functional requirements, where pressure control may be maintained. These results indicated a press should be able to apply a range of pressures estimated greater than 0.5 psi. This value was established from the lowest measured force applied by the tortilla press. The 25 pounds of force over 50 square inches corresponded to about 0.5 psi.

Because the porcine liver tissue was determined to be acceptable, this pressure can be used as a minimum in the porcine liver tissue processing to form the regenerative tissue material. The pressure should be easily adjustable to accommodate different thicknesses of porcine liver tissue samples. The press used for the process should be equipped with a timer that allows the user to set a duration or hold time of the press cycle. Any press used for the process should be able to handle a range of sample sizes, estimated at a minimum of 256 square inches.

For current liver slice processing with a dough press, the working area can be decreased to a 10 inch by 16 inch area. This type of change may result in an increase in pressure to about 0.83 psi with the current force applied at 132.6 pounds, and the desired pressure maintained evenly across all samples of the porcine liver tissue.

A press used for the process may be used on porcine liver tissues having varying degrees of thickness, shapes, and consistencies due to vascularity, and should be equipped with features that allow for the preparation of samples on removable trays. Fluid drainage channels may be machined into the platen to allow fluid to collect in a desired location rather than spilling over onto a table. Appropriate safety features should be used.

An example press found to be acceptable is the AIM Joraco clean room press with a plate size of about 24 inches by 20 inches to accommodate slice expansion at multiple presses. An example AIM Joraco press is a clean room press used in pharmaceutical, life science, and regenerative medicines, such as the AIM Joraco press that is clean room compliant equivalent to class 100, has a quiet operation with noise level reduction of 40 dB (A) or more, stainless steel construction, and press force monitoring and real-time data logging with computer processing capability. The press may be implemented to meet the requirements for the various pressures, fluid temperatures, and air cleanliness.

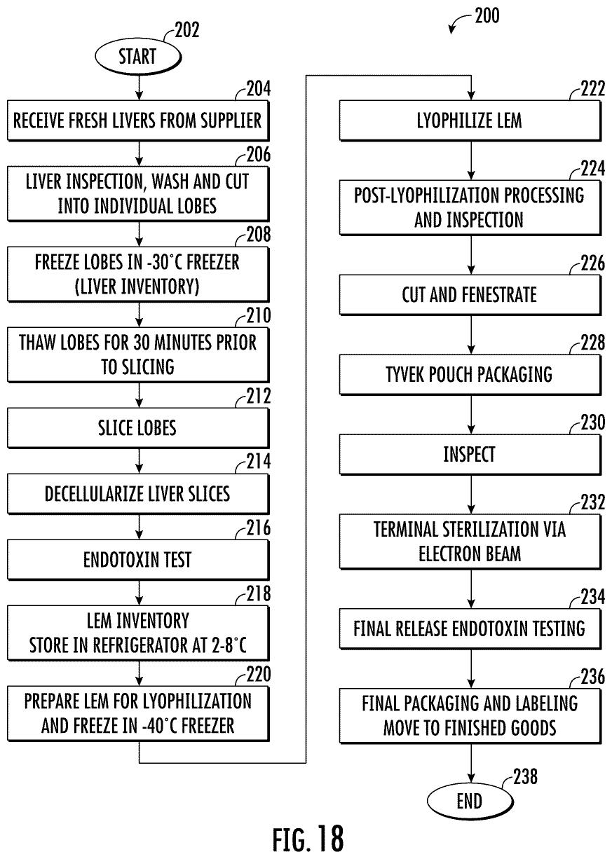

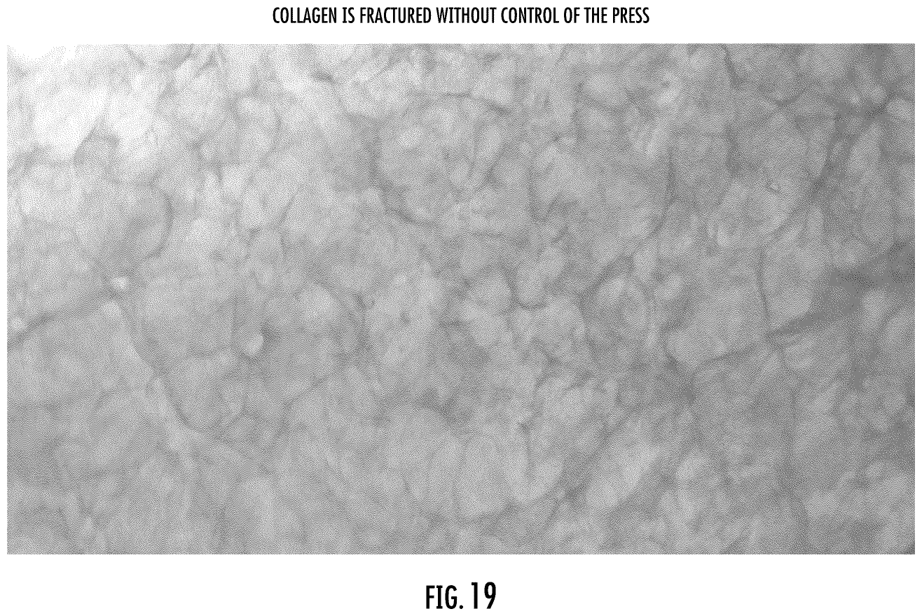

Referring now to FIG. 18, there is illustrated a high-level flowchart at 200 showing the complete start to finish production, inspection, and testing of the regenerative tissue repair product derived from the extracellular matrix of decellularized animal liver such as porcine liver. In this process flowchart, the process starts (Block 202) and fresh livers are received from a supplier (Block 204), which are then inspected, washed, and cut into individual lobes (Block 206). The lobes are frozen in a −30° C. freezer for liver inventory in this example (Block 208). Lobes are thawed for 30 minutes prior to slicing (Block 210). The lobes are sliced (Block 212) and the liver slices are decellularized in an example process as described before (Block 214).

An endotoxin test is performed (Block 216) and the regenerative tissue repair product is inventoried and stored at about 2°° C. to about 8° C. (Block 218). The “LEM” refers to the same product as Liver Extracellular Matrix. The regenerative tissue repair product is then prepared for lyophilization and frozen in a −40° C. freezer (Block 220). The regenerative tissue repair product is lyophilized (Block 222), followed by post-lyophilization processing and inspection (Block 224). The regenerative tissue repair product is then cut and fenestrated for openings (Block 226) and packaged in a Tyvek pouch (Block 228) and inspected (Block 230). The regenerative tissue repair product is then sterilized as a terminal step via electron beam sterilization (Block 232) and a final release endotoxin testing performed (Block 234) with final packaging and labeling, and moved to a finished goods inventory (Block 236). The process ends (Block 238).

Additional details concerning pressing of the liver slices was obtained, allowing greater control of the pressing operation. As noted before, the pressing operation aids in expelling fluid from the tissue and reabsorption of deionized water or the washing solution in the subsequent rinses.

A clean room compliant Toggle-Aire Model 2530, Benchtop Pneumatic Toggle Press was implemented with custom tooling specific to RTT Medical's decellularization pressing application. The press operates by compressed air, is rated for a clean room environment, and includes Force Monitoring controls for better consistency during decellularization. All contact material is stainless steel.

The Toggle-Aire Model 2530 is a bench-mounted pneumatic toggle press designed to operate in highly controlled clean room environments. This specific unit has been adapted with custom tooling designed to accommodate RTT Medical's proprietary decellularization process.

The press operates using a compressed air system, which drives the toggle mechanism to apply consistent pressure on the material during the decellularization process. This pneumatic operation allows for precise force application without the variability associated with manual presses.

The pneumatic press is compliant with clean room standards, meaning it minimizes particle generation and contamination, making it suitable for medical manufacturing environments.

Integrated force monitoring controls are a critical aspect of the decellularization process. These controls provide real-time feedback on the force applied during pressing, ensuring that the operation remains within predefined limits. This enhances consistency and precision, which is essential for preserving the integrity of the biological material being decellularized.

To meet both clean room and medical-grade standards, all components of the press that come into direct contact with the liver tissue material are constructed from stainless steel. This material ensures high durability, corrosion resistance, and ease of sterilization, which are essential for maintaining the sterility and integrity of the decellularized tissues.

The pressing surface is 15.5″×17.5″ (271.25 in²) to accommodate a 300 gram jar of material during decellularization, which is the standard jar size used in RTT Medical's decellularization process.

The size of the pressing surface was chosen specifically to handle the volume and dimensions of the material container used during the pressing operation. The jar is placed on the pressing platen, and the press uniformly applies force across the entire surface to ensure even decellularization of the biological material contained within.

The pneumatic press is rated for a maximum compressive force of 20,000 pounds (lbs.), which reflects its full capacity under optimal operating conditions. This force is more than sufficient for most decellularization applications but may not be necessary for routine operations.

Through preliminary testing and process development, it was determined that operating the press at 60% of its maximum capacity, equivalent to 12,000 pounds, significantly reduces the mechanical stress on both the press and the material being processed. By limiting the force to this level, the risk of long-term wear and damage to the press is minimized, extending its operational lifespan and reducing the likelihood of equipment failure during critical decellularization tasks.

Operating at 60% capacity also helps protect the material being processed from excessive force, which could lead to undesirable changes in the tissue structure or compromise the quality of the decellularized product.

Previous decellularization trials using a manual press established that a consistent pressure of 0.5 PSI was optimal for the pressing operation. This pressure was found to be sufficient for decellularizing tissue material without causing damage or altering the material properties.

As the development progressed to the pneumatic press, this 0.5 PSI pressure was maintained as the minimum required pressure within the operating window. The pneumatic press allows for precise control over the initial force applied to the material, making it possible to replicate and enhance the results achieved with the manual press.

In order to achieve a pressure of 0.5 PSI on the pressing surface of 271.25 square inches, the initial force parameter on the pneumatic press was set to 150 pounds. This precise force setting ensures that the desired pressure is consistently applied at the start of each decellularization cycle.

During setup and testing, it was observed that setting the initial force parameter higher than 350 pounds could cause the force applied during pressing to spike beyond the safe operating capacity of 12,000 pounds. These force spikes posed a risk of overloading the material and the press itself, potentially leading to equipment damage or compromised material integrity.

As a result, the upper limit of the operating window was set based on these findings, ensuring that the initial force applied does not exceed 350 pounds. This constraint helps prevent unintended force spikes, ensuring the pressing operation remains within the safe and optimal range for decellularization.

In an example trial, liver slices were pressed with a starting pressure setpoint of 150 ±25 pounds to 350 ±25 pounds. The preferred pressure setpoint for Pre-Triton, Post-Triton, and Pre-Sodium was found to be 250 ±25 pounds. The term Triton refers to the Triton detergent solution described above and the sodium as the sodium hydroxide (NaOH) solution described above. The pressing study results are now described.

The recorded pressure setpoint for Pre-Triton high pressure was 363 pounds. The average jar weight before the press was 325.73 grams. The average jar weight after the press was 177.8 grams. This is an average weight decrease of 45.54% with an average pressure output of 8971.66 pounds.

The recorded pressure setpoint for Pre-Triton low pressure was 167 pounds. The average jar weight before the press was 320.60 grams. The average jar weight after the press was 176.95 grams. This was an average weight decrease of 44.81% with an average pressure output of 8650.66 pounds.

The recorded pressure setpoint for Post-Triton high pressure was 353 pounds. The average jar weight before the press was 188.84 grams. The average jar weight after the press was 95.63 grams. This was an average weight decrease of 49.36% with an average pressure output of 4753.55 pounds.

The recorded pressure setpoint for Post-Triton low pressure was 168 pounds. The average jar weight before the press was 169.92 grams. The average jar weight after the press was 100.27 grams. This was an average weight decrease of 40.99% with an average pressure output of 3286.77 pounds.

With added press, a pre-sodium press was not a requirement during decellularization and may be performed at the discretion of the operator if there are remaining cells in the material. The pressure outputs at this cycle were not recorded at the high and low ends of the operating window. This cycle was pressed at a nominal setpoint of 255 pounds. The average jar weight before the press was 146.00 grams. The average jar weight after the press was 64.91 grams. This is an average weight decrease of 55.54%. Adding the control and consistency of the press has improved decellularization in the previous steps and this last press rarely required. There was also a lower amount of fracturing of the collagen matrix, and the collagen consistency has improved.

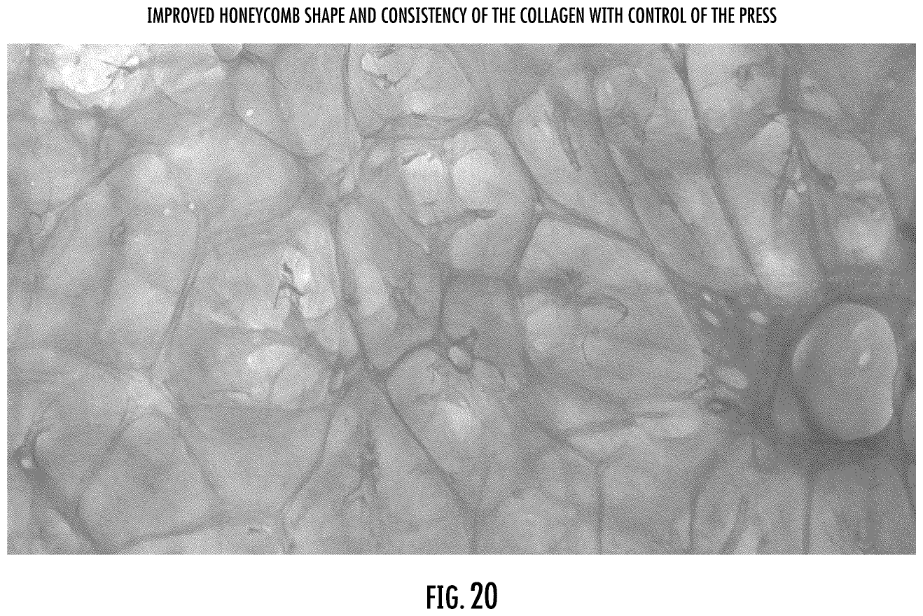

FIG. 19 is an image of the collagen fractured without control of the press, while FIG. 20 is an example with the press control as described and showing the improved honeycomb shape and consistency of the collagen with control of the press.

All samples processed under high pressure parameters resulted in a DNA/weight of product well below the limit of 783 ng/mg corresponding to the competition limit. The average DNA of the regenerative tissue repair product is 167 ng/mg.

All samples processed under low pressure parameters resulted in a DNA/weight of product well below the limit of the competition limit. The average DNA of jars 10-18 was 719.22 ng/mg.

Collagen content was measured in mg/ml. The average soluble collagen content for high pressure samples (jars 1-9) measured 10.96 mg/ml. The average soluble collagen content for low pressure samples (jars 10-18) measured 16.21 mg/ml.

Protein content was measured in μg/ml. The average protein content for high pressure samples (jars 1-9) measured 120.01 μg/ml. The average protein content for low pressure samples (jars 10-18) measured 138.06 μg/ml.

It is possible to use the lower pressure on the press to maintain low DNA, increase collagen and protein levels, and ultimately improve the consistency of the final collagen device as the regenerative tissue repair product.

Many modifications and other embodiments of the invention will come to the mind of one skilled in the art having the benefit of the teachings presented in the foregoing descriptions and the associated drawings. Therefore, it is understood that the invention is not to be limited to the specific embodiments disclosed, and that modifications and embodiments are intended to be included within the scope of the appended claims.

1. A regenerative tissue repair product, comprising:
a decellularized extracellular matrix derived from animal liver; and
the decellularized extracellular matrix retaining the collagen morphology of the animal liver prior to decellularization and having less than 500 ng DNA/mg dry extracellular matrix and greater than 760 μg total collagen per milligram of dry extracellular matrix, the collagen comprising soluble collagen and insoluble collagen.

2. The regenerative tissue repair product of claim 1, wherein the decellularized extracellular matrix has greater than 1.6 μg glycosaminoglycans per milligram of dry extracellular matrix and greater than 150 μg elastin per milligram of dry extracellular matrix.

3. The regenerative tissue repair product of claim 1, wherein the decellularized extracellular matrix is configured into a single or multi-layer sheet.

4. The regenerative tissue repair product of claim 3, wherein the sheet formed from the decellularized extracellular matrix is about 0.5 mm to 3.2 mm thick.

5. The regenerative tissue repair product of claim 1, wherein the decellularized extracellular matrix retains the natural reticular, honeycomb morphology of the animal liver prior to decellularization.

6. The regenerative tissue repair product of claim 5, wherein the decellularized extracellular matrix retains substantially the amount of collagen and proteins associated with the extracellular matrix before decellularization to recruit stem cells and promote optimal cellular growth in surrounding tissues when applied onto tissue to aid in healing tissue to be repaired.

7. The regenerative tissue repair product of claim 1, wherein the animal liver comprises a porcine liver.

8. The regenerative tissue repair product of claim 1, wherein the decellularized extracellular matrix is configured as a powder.

9. A regenerative tissue repair product, comprising:
a decellularized extracellular matrix derived from animal liver, said decellularized extracellular matrix retaining the reticular, honeycomb and collagen morphology of the animal liver prior to decellularization; and
the decellularized extracellular matrix having less than 500 ng DNA/mg dry extracellular matrix, greater than 1.6 μg glycosaminoglycans per milligram of dry extracellular matrix, greater than 760 μg collagen per milligram of dry extracellular matrix, the collagen comprising soluble collagen and insoluble collagen, and greater than 150 μg elastin per milligram of dry extracellular matrix, and greater than 110 mg/ml of protein to recruit stem cells and promote optimal cellular growth in surrounding tissues when applied onto tissue to aid in healing tissue to be repaired.

10. The regenerative tissue repair product of

9., wherein the decellularized extracellular matrix is configured into a single or multi-layer sheet.

11. The regenerative tissue repair product of claim 10, wherein the sheet formed from the decellularized extracellular matrix is about 0.5 mm to 3.2 mm thick.

12. The regenerative tissue repair product of claim 9, wherein the animal liver comprises a porcine liver.

13. The regenerative tissue repair product of claim 9, wherein the decellularized extracellular matrix is configured into a powder.

14. A method of forming a regenerative tissue repair product, comprising:
slicing an animal liver into a liver slice; and
pressing and rinsing the liver slice to form a decellularized extracellular matrix from the liver slice that retains the collagen morphology of the animal liver prior to decellularization and has less than 500 ng DNA/mg dry extracellular matrix, greater than 760 μg collagen per milligram dry extracellular matrix, the collagen comprising soluble collagen and insoluble collagen, greater than 1.6 μg glycosaminoglycans per milligram of dry extracellular matrix, and greater than 150 μg elastin per milligram dry extracellular matrix.

15. The method of claim 14, comprising pressing and rinsing the liver slice multiple times.

16. The method of claim 15, comprising:
a) pressing the liver slice a first time;
b) exposing the pressed liver slice to a detergent solution to aid in breaking up the cells within the liver slice; and
c) pressing the liver slice a second time.

17. The method of claim 16, wherein the first pressing occurs at a higher pressure than the second pressing.

18. The method of claim 16, wherein the first pressing occurs at a time period longer than the second pressing.

19. The method of claim 16, comprising pressing the liver slice a third time at a pressure lower than the pressures of the first and second pressings.

20. The method of claim 19, comprising exposing the liver slice to a higher pH rinsing after the third pressing to facilitate an osmolality change to aid in removing cellular debris.

21. The method of claim 14 comprising pressing the liver slice within a press having a mesh configured to allow fluid and cellular debris to move away from the liver slice.

22. The method of claim 14, wherein the decellularized extracellular matrix is configured into a single or multi-layer sheet.

23. The method of claim 14, wherein the sheet formed from the decellularized extracellular matrix is about 0.5 mm to 3.2 mm thick.

24. The method of claim 14, wherein the decellularized extracellular matrix retains the natural reticular, honeycomb morphology of the animal liver prior to decellularization.

25. The method of claim 24, wherein the decellularized extracellular matrix retains substantially the amount of collagen and proteins associated with the extracellular matrix before decellularization to recruit stem cells and promote optimal cellular growth in surrounding tissues when applied onto tissue to aid in healing tissue to be repaired.

26. The method of claim 14, wherein the animal liver comprises a porcine liver.

27. The method of claim 14, wherein the decellularized extracellular matrix is configured into a powder.

28. The method of claim 15, wherein the decellularized extracellular matrix has greater than 110 mg/ml of protein to recruit stem cells and promote optimal cellular growth in surrounding tissues when applied onto tissue to aid in healing tissue to be repaired.
